(19) United States
(12) Patent Application Publication
(10) Pub. No.: US 2022/0287935 A1
(43) Pub. Date: Sep. 15, 2022

(54) CANNABINOID-CONTAINING SKIN CARE COMPOSITIONS

(71) Applicant: Cannasmetics Ltd., Petach Tikva (IL)

(72) Inventors: Ben Yitzhak YELLIN, Kokhav Yair Tzur Yigal (IL); Nava RAVID, Herzlia (IL)

(73) Assignee: Cannasmetics Ltd., Petach Tikva (IL)

(21) Appl. No.: 17/690,084

(22) Filed: Mar. 9, 2022

Related U.S. Application Data
(60) Provisional application No. 63/160,018, filed on Mar. 12, 2021.

(51) Int. Cl. A61K 8/34; A61K 8/92; A61Q 19/00

(52) U.S. Cl. CPC A61K 8/347; A61K 8/922; A61Q 19/00

(57) ABSTRACT

Cosmetic or cosmeceutical formulations comprising cannabidiol (CBD), cannabigerol (CBG) and pomegranate seed oil (PSO), products containing same and uses thereof are provided.

### RELATED APPLICATION

This application claims the benefit of priority under 35 USC § 119(e) of U.S. Provisional Patent Application No. 63/160,018 filed on Mar. 12, 2021, the contents of which are incorporated herein by reference in their entirety.

### FIELD AND BACKGROUND OF THE INVENTION

The present invention, in some embodiments thereof, relates to skin care and, more particularly, but not exclusively, to cannabinoid-containing compositions, to skin care, cosmetic and cosmeceutical products containing same and to uses thereof in treating or reducing damage to a keratinous tissue.

The cannabis plant has many naturally occurring substances that are of great interest in the fields of science and medicine. Isolated compounds from the cannabis plant are collectively referred to herein and in the art as cannabinoids, and include, inter alia, ^{Δ}9-tetrahydrocannabinol (THC), cannabidiol (CBD), cannabichromene (CBC), cannabigerol (CBG), cannabinol (CBN), cannabidivarin (CBDV). While THC has psychoactive effects, CBD, CBC, CBG, and CBDV do not.

CBG is a non-psychoactive cannabinoid found in the cannabis plant. All cannabinoids in the early stage of the cannabis plant's life begins as CBG. CBG is found in higher concentrations in hemp plants as opposed to marijuana plants that are grown to have higher concentrations of tetrahydrocannabinol (THC).

Cannabinoids can be isolated by extraction or cold pressing from cannabis plants. Plants in the cannabis genus from which cannabinoids can be extracted include Cannabis sativa, Cannabis ruderalis, and Cannabis indica. Cannabinoids can also be synthetically prepared (synthesized).

Cosmetics are body-care substances used to enhance the human body's appearance or odor. Cosmetics are used to cleanse, beautify, promote attractiveness, and alter appearance without affecting the body's structure or function. Various types of cosmetics exist. Generally, they are cosmetics applied to the skin (face and body), cosmetics applied to the lips, cleansing cosmetics (soap, shampoo, and conditioner), cosmetics applied to change odor (perfume, body mist, deodorant), and cosmetics applied to the eyes (mascara, eyebrow pencil).

Cosmetics applied to the skin or any other keratinous tissue, whether stay-on or rinse-off, are typically intended to exhibit anti-microbial, anti-inflammatory, anti-oxidant, anti-aging, and/or wound healing properties. Various substances are added to cosmetics to achieve these effects, and continuous efforts are still made to identify substances with these properties to be used in cosmetics.

U.S. Patent Application Publication No. 2020/0345656 discloses non-aqueous cannabinoid-containing compositions for application to bodily cavities. U.S. Patent Application Publication No. 2018/0264121 discloses cosmetic compositions containing CBD in an oily solvent.

U.S. Patent Application Publication Nos. 2016/0235661 and 2020/0188325 disclose topical compositions for dermal applications containing CBD, CBG, an anti-microbial agent, an anti-inflammatory agent and an anti-oxidant.

### SUMMARY OF THE INVENTION

According to an aspect of some embodiments of the present invention there is provided a cosmetic or cosmeceutical composition comprising, or consisting of, an isolated cannabidiol (CBD), an isolated cannabigerol (CBG) and pomegranate seed oil (PSO), as described herein in any of the respective embodiments and any combination thereof.

According to an aspect of some embodiments of the present invention there is provided a cosmetic or cosmeceutical formulation comprising the composition as described herein in any of the respective embodiments and any combination thereof. According to some of any of the embodiments described herein, the formulation further comprises a cosmetically and/or cosmeceutically acceptable carrier.

According to some of any of the embodiments described herein, the formulation is in a form of a cream, an ointment, a paste, a gel, a lotion, a milk, an oil, a suspension, a solution, an aerosol, a spray, a foam, a powder (e.g., a pressed powder or a loose powder), a serum or a mousse.

According to some of any of the embodiments described herein, a concentration of the composition ranges from 0.1 to 20, % by weight of the total weight of the formulation.

According to an aspect of some embodiments of the present invention there is provided a cosmetic or cosmeceutical formulation comprising an isolated cannabidiol (CBD), an isolated cannabigerol (CBG) and pomegranate seed oil (PSO).

According to some of any of the embodiments described herein, a concentration of the PSO in the formulation is no more than 1,000-folds, or no more than 500-folds, or no more than 100-folds, or no more than 50-folds, the concentration of the CBD or CBG.

According to an aspect of some embodiments of the present invention there is provided a cosmetic or cosmeceutical formulation comprising cannabidiol (CBD), an isolated cannabigerol (CBG) and pomegranate seed oil (PSO), wherein a concentration of the PSO in the composition is no more than 1,000-folds, or no more than 500-folds, or no more than 100-folds, or no more than 50-folds, the concentration of the CBD or CBG.

According to some of any of the embodiments described herein, the CBD is an isolated CBD.

According to some of any of the embodiments described herein, the CBG is an isolated CBG.

According to some of any of the embodiments described herein, an amount of PSO ranges from 0.01 to 5, or from 0.1 to 4, or from 0.1 to 2, % by weight of the total weight of the formulation.

According to some of any of the embodiments described herein, an amount of CBD ranges from 0.1 to 2, % by weight of the total weight of the composition.

According to some of any of the embodiments described herein, an amount of CBG ranges from 0.01 to 2, or from 0.01 to 1, % by weight of the total weight of the composition.

According to some of any of the embodiments described herein, a weight ratio of CBD to PSO ranges from 2:1 to 1:100, or from 2:1 to 1:50, or from 1:1 to 100:1.

According to some of any of the embodiments described herein, a weight ratio of CBG to PSO ranges from 1:5 to 1:100, or from 1:5 to 1:50.

According to some of any of the embodiments described herein, a weight ratio of CBD to CBG ranges from 10:1 to 1:1, or from 10:1 to 2:1. According to some of any of the embodiments described herein, a weight ratio of CBD and CBG ranges from 2:1 to 10:1 and a weight ratio of PSO and CBD ranges from 1:5 to 50:1, or from 1:2 to 20:1.

According to some of any of the embodiments described herein, the formulation further comprises a cosmetically and/or cosmeceutically acceptable carrier. According to some of any of the embodiments described herein, the cosmetic or cosmeceutical formulation is in a form of a cream, an ointment, a paste, a gel, a lotion, a milk, an oil, a suspension, a solution, an aerosol, a spray, a foam, a powder (e.g., a pressed powder or a loose powder), a serum or a mousse.

According to some of any of the embodiments described herein, a total concentration of the PSO, CBD and CBG ranges from 0.1 to 50, or from 1 to 50, % by

According to an aspect of some embodiments of the present invention there is provided a cosmetic or cosmeceutical product comprising the composition as described herein in any of the respective embodiments and any combination thereof, or the formulation as described herein in any of the respective embodiments and any combination thereof.

According to some of any of the embodiments described herein, the product is a skin care product, a make-up product (including eye shadows, make-up, lipstick, lacquer, etc.), a men's grooming products, a sunscreen product, a female hygiene product, an oral cavity-care product or a hair care product. According to some of any of the embodiments described herein, the product is an anti-aging product, an anti-acne product, an anti-diaper rush product, a shampoo, a body lotion, a body cream, a face cream, a face lotion, a face mask, a body and/or face wash product, a cleansing product (for hair and/or skin and/or mucosal tissue such as oral cavity or vagina), a hygienic product, an anti-pigmentation product, a make-up product, an anesthetic product, a sunscreen product.

According to an aspect of some embodiments of the present invention there is provided a method of treating, reducing or preventing (protecting from) a damage to a keratinous tissue or mucosal tissue, the method comprising topically applying to the keratinous tissue or the mucosal tissue the composition as described herein in any of the respective embodiments and any combination thereof, the formulation as described herein in any of the respective embodiments and any combination thereof, or the product as described herein in any of the respective embodiments and any combination thereof. According to an aspect of some embodiments of the present invention there is provided a composition, a formulation or a product, as described herein in any of the respective embodiments and any combination thereof, for use in treating, reducing or preventing (protecting from) a damage to a keratinous tissue or mucosal tissue.

According to some of any of the embodiments described herein, the keratinous tissue is a facial tissue, a hair tissue, or a skin tissue.

According to some of any of the embodiments described herein, the mucosal tissue is a vaginal tissue.

According to some of any of the embodiments described herein, the mucosal tissue is in the oral cavity. According to some of any of the embodiments described herein, the damage is associated with aging.

According to some of any of the embodiments described herein, the damage is associated with a dermatological condition.

According to some of any of the embodiments described herein, the damage is associated with skin inflammation.

According to some of any of the embodiments described herein, the dermatological condition is selected from atopic dermatitis, psoriasis, pigmentation, acne, eczema, xerosis, ichthyosis, keratosis, keratoderma, pruritus, dermatitis, neuro-dermatitis, dermatitis herpetiformis, actinic keratosis, hyper and inflamed keratosis, atopic eczema, melanoma, rosacea, urticaria, seborrheic dermatitis, skin cancer, and xeroderma pigmentosum.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A, 1B, 1C and 1D are bar graphs showing the effect of various concentrations of CBD or CBG (FIG. 1A); of various concentrations of PSO (FIG. 1B);

of combination samples 1-5 as described in Example 1 (Table 1) (FIG. 1C); and of combinations samples 6-10 as described in Example 1 (Table 1) (FIG. 1D), on THP-1 cells viability.

FIGS. 2A-2B are bar graphs showing the effect of various concentrations of CBD, CBG and PSO on TNFα secretion from LPS-stimulated THP-1 cells, presented the percentage of the control (FIG. 2A) and as the ratio between the cells' viability values and the TNFα secretion values, for a same given well (FIG. 2B).

FIGS. 3A-3B are bar graphs showing the effect of combination samples 1-5 as described in Example 1 (Table 1) on TNFα secretion from LPS-stimulated THP-1 cells, presented the percentage of the control (FIG. 3A) and as the ratio between the cells' viability values and the TNFα secretion values, for a same given well (FIG. 3B).

FIGS. 4A-4B are bar graphs showing the effect of combination samples 6-10 as described in Example 1 (Table 1) on TNFα secretion from LPS-stimulated THP-1 cells, presented the percentage of the control (FIG. 4A) and as the ratio between the cells' viability values and the TNFα secretion values, for a same given well (FIG. 4B).

FIGS. 5A-5B are bar graphs showing the effect of various concentrations of CBD, CBG and PSO on IL6 secretion from LPS-stimulated THP-1 cells, presented the percentage of the control (FIG. 5A) and as the ratio between the cells' viability values and the TNFα secretion values, for a same given well (FIG. 5B).

FIGS. 6A-6B are bar graphs showing the effect of combination samples 1-5 as described in Example 1 (Table 1) on IL6 secretion from LPS-stimulated THP-1 cells, presented the percentage of the control (FIG. 6A) and as the ratio between the cells' viability values and the TNFα secretion values, for a same given well (FIG. 6B).

FIGS. 7A-7B are bar graphs showing the effect of combination samples 6-10 as described in Example 1 (Table 1) on IL6 secretion from LPS-stimulated THP-1 cells, presented the percentage of the control (FIG. 7A) and as the ratio between the cells' viability values and the TNFα secretion values, for a same given well (FIG. 7B).

FIGS. 8A, 8B and 8C are bar graphs showing the effect of various concentrations of CBD, CBG and PSO (FIG. 8A), of combination samples 1-5 as described in Example 1 (Table 1) (FIG. 8B); and of combinations samples 6-10 as described in Example 1 (Table 1) (FIG. 8C), on viability of HaCat cells.

FIGS. 9A, 9B and 9C are bar graphs showing the effect of various concentrations of CBD, CBG and PSO (FIG. 9A), of combination samples 1-5 as described in Example 1 (Table 1) (FIG. 9B); and of combinations samples 6-10 as described in Example 1 (Table 1) (FIG. 9C), on ROS production from HaCat cells in the presence of H₂O₂.

### DESCRIPTION OF SPECIFIC EMBODIMENTS OF THE INVENTION

The present invention, in some embodiments thereof, relates to skin care and, more particularly, but not exclusively, to cannabinoid-containing compositions, to skin care, cosmetic and cosmeceutical products containing same and to uses thereof in treating or reducing damage to a keratinous tissue.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have sought for cosmetic/cosmeceutical compositions that are made essentially from biomaterials, namely, active ingredients that are derived from natural sources, yet exhibit properties such as anti-oxidation, anti-inflammation and/or antibacterial activity, and can be used in treating, reducing or preventing damage to keratinous tissues (e.g., skin tissues) and/or mucosal tissues.

The present inventors have envisioned that combining non-psychoactive cannabinoids and a natural oil may impart the desired properties when applied to skin and thereby act as “bio-defense” compositions for treating, reducing or preventing skin damage and/or improving the appearance and condition of the skin and other keratinous tissues.

The present inventors have selected CBD, CBG and PSO and have studied the effect of each of these components alone, and of varying combinations thereof on the viability, secretion of inflammation markers and ROS production, of cells, and have uncovered that certain combinations of these natural components exhibit anti-inflammatory and anti-oxidation activities without affecting cells viability.

Embodiments of the present invention therefore relate to novel cosmetic and/or cosmeceutical compositions containing CBD, CBG and PSO; to cosmetic and/or cosmeceutical formulations comprising same and to uses of the compositions or formulations for making up cosmetic and/or cosmeceutical products (e.g., skin-care products).

As used herein throughout, the term “cosmetic” with regard to any of the compositions and methods (and any other aspect) disclosed herein, describes compositions which enhance the appearance or odor of the human body.

In some embodiments, the term “cosmetic” follows the definition regulatory authorities worldwide. For example, in some embodiments, the term “cosmetic” follows the definition of the U.S. Food and Drug Administration (FDA), as being “intended to be applied to the human body for cleansing, beautifying, promoting attractiveness, or altering the appearance without affecting the body's structure or functions”.

As used herein, the phrase “cosmeceutical composition” describes a composition for topical administration as a cosmetic, which comprises a biologically active ingredient, that is, a compound that exhibits a biological activity such as, for example, anti-inflammatory, anti-bacterial and/or anti-oxidation activity.

The phrase “keratinous material” or “keratinous substrate” or “keratinous tissue”, which are used interchangeably herein, means, in some embodiments of the present invention, a material, substrate or tissue that is enriched with keratin, including, for example, nails, hair and skin, and especially bodily areas like the face, cheeks, hands, body, legs, around the eyes, the eyelids and the lips.

“Skin” means the outermost protective covering of mammals that is composed of cells such as keratinocytes, fibroblasts and melanocytes. Skin includes an outer epidermal layer and an underlying dermal layer. Skin may also include hair and nails as well as other types of cells commonly associated with skin, such as, for example, myocytes, Merkel cells, Langerhans cells, macrophages, stem cells, sebocytes, nerve cells and adipocytes.

Compositions:

According to an aspect of some embodiments of the present invention there is provided a cosmetic and/or cosmeceutical composition which comprises CBD, CBG and pomegranate seed oil (PSO).

According to some embodiments, the CBD is an isolated material, which is also referred to herein as “isolated CBD”, that is, it is a compound per se, which does not form a part of a composition such as, for example, an oil composition or an oily plant extract. The CBD can be synthetically prepared or isolated from a cannabis plant (e.g., as described in the Examples section that follows). The CBD can be obtained from commercial vendors (a commercially available product). In some embodiments, the isolated CBD comprises at least 90%, or at least 95%, or at least 99%, or at least 99.5%, or 100% CBD.

According to some embodiments, the CBG is an isolated material, which is also referred to herein as “isolated CBG”, that is, it is a compound per se, which does not form a part of a composition such as, for example, an oil composition or an oily plant extract. The CBG can be synthetically prepared or isolated from a cannabis plant (e.g., as described in the Examples section that follows). The CBG can be obtained from commercial vendors (a commercially available product). In some embodiments, the isolated CBG comprises at least 90%, or at least 95%, or at least 99%, or at least 99.5%, or 100% CBG.

According to some embodiments, the PSO is a commercially available product.

According to some of any of the embodiments described herein, the cosmetic or cosmeceutical composition comprises isolated CBD, as defined herein, isolated CBG, as defined herein, and PSO.

According to some of any of the embodiments described herein, a concentration of the PSO in the composition is no more than 1,000-folds, or no more than 500-folds, or no more than 100-folds the concentration of the CBD and/or CBG. For example, a concentration of PSO can be, for example, 2-folds, 3-folds, 5-folds, 10-folds, 20-folds, 30-folds, 40-folds, 50-folds, 60-folds, 70-folds, 80-folds, 90-folds, or 100-folds, or, optionally, 200-folds, 300-folds, 400-folds, or 500-folds, or 600-folds, the concentration of the CBD and/or CBG, or, further optionally, can be 0.1-folds, 0.2-folds, 0.3-folds, 0.4-folds, 0.5-folds, 0.6-folds, 0.7-folds, 0.8-folds, 0.9-folds, or 1-folds, the concentration of the CBD and/or CBG, in the composition.

According to some of any of the embodiments described herein, a concentration ratio of the PSO to CBD in the composition ranges from 1:10 to 1000:1, or from 1:10 to 100:1, or from 1:3 to 10:1, or from 1:3 to 1:1, including any intermediate values and subranges therebetween.

According to some of any of the embodiments described herein, an amount of PSO ranges from 0.001 to 50, or from 0.1 to 40, or from 0.1 to 20, % by weight of the total weight of the composition, including any intermediate values and subranges therebetween. According to some embodiments, an amount of PSO ranges from 0.01 to 10, or from 0.01 to 5, or from 0.1 to 5, or from 0.1 to 0.2, % by weight of the total weight of the composition, including any intermediate values and subranges therebetween. According to some embodiments, an amount of PSO ranges from 1 to 50, or from 1 to 40, or from 1 to 30, or from 5 to 25, % by weight of the total weight of the composition, including any intermediate values and subranges therebetween.

According to some of any of the embodiments described herein, an amount of the CBD ranges from 10 to 70, or from 20 to 70, or from 25 to 70, or from 30 to 70, or from 30 to 60, or from 40 to 60, or from 30 to 50, or from 20 to 50, or from 20 to 30, % by weight of the total weight of the composition, including any intermediate values and subranges therebetween.

According to some of any of the embodiments described herein, an amount of the CBG ranges from 20 to 80, or from 30 to 80, or from 30 to 70, or from 30 to 60, or from 30 to 50, or from 40 to 50, or from 40 to 60, % by weight of the total weight of the composition.

According to some of any of the embodiments described herein, a weight ratio of CBD to PSO ranges from 1:2 to 1000:1, or from 1:1 to 500:1, or from 1:1 to 100:1, including any intermediate values and subranges therebetween.

According to some of any of the embodiments described herein, a weight ratio of CBG to PSO ranges from 1:2 to 1000:1, or from 1:1 to 700:1, or from 1:1 to 100:1, including any intermediate values and subranges therebetween.

According to some of any of the embodiments described herein, a weight ratio of CBD to CBG ranges from 3:1 to 1:3, or from 1:1 to 3:1, or from 1:1 to 2:1, including any intermediate values and subranges therebetween.

According to some of any of the embodiments described herein, an amount of CBG is at least 30, or at least 40, or at least 50%, by weight of the total weight of the composition.

According to some of any of the embodiments described herein, an amount of CBD is no more than 50% by weight of the total weight of the composition.

According to some of any of the embodiments described herein, a concentration ratio of CBG and CBG ranges from 3:1 to 1:3 or from 1:1 to 3:1 or from 1:1 to 2:1, including any intermediate values and subranges therebetween.

According to some of any of the embodiments described herein, a concentration ratio of CBD and CBG is about 1:2.

According to some of any of the embodiments described herein, a concentration ratio of PSO and CBD ranges from 50:1 to 100:1, or from 60:1 to 100:1, or from 60:1 to 80:1, including any intermediate values and subranges therebetween.

According to some of these embodiments, a concentration ratio of CBD to CBG is about 1:1.

According to some of any of the embodiments described herein, a concentration ratio of CBD or CBG to PSO ranges from 1:1 to 10:1, or from 1:1 to 5:1, including any intermediate values and subranges therebetween, or is about 3:1.

According to some of any of the embodiments described herein, a weight ratio of CBD to CBG is about 1:1 and a weight ratio of PSO to CBD or CBG ranges from 1:1 to 1:50, or from 1:20 to 1:50, or from 1:30 to 1:40, including any intermediate values and subranges therebetween.

According to some of any of the embodiments described herein, a weight ratio of CBD, CBG and PSO is about 1:1:1.

According to some of any of the embodiments described herein, a weight ratio of CBD and CBG is 1:2 and a weight ratio of PSO and CBD ranges from 1:1 to 2:1, including any intermediate values and subranges therebetween.

According to some of any of the embodiments described herein, the cosmetic or cosmeceutical composition comprises from 0.1 to 3, or from 0.1 to 2, or from 0.5 to 2, mg CBD (e.g., isolated CBD), including any intermediate values and subranges therebetween.

According to some of any of the embodiments described herein, the cosmetic or cosmeceutical composition comprises from 0.1 to 5, or from 0.1 to 4, or from 0.5 to 3.5, mg CBG (e.g., isolated CBG), including any intermediate values and subranges therebetween.

According to some of any of the embodiments described herein, the cosmetic or cosmeceutical composition comprises from 0.001 to 2 mg PSO, including any intermediate values and subranges therebetween.

According to some of any of the embodiments described herein, the cosmetic or cosmeceutical composition comprises from 1 to 2 mg CBD; from 3 to 4 mg CBG; and from 1 to 2 mg PSO.

According to some of any of the embodiments described herein, the cosmetic or cosmeceutical composition comprises from 0.5 to 1 mg CBD; from 0.5 to 1 mg CBG; and from 0.001 to 0.005, or from 0.002 to 0.003 mg PSO.

Formulations and Products:

According to an aspect of some embodiments of the present invention there is provided a cosmetic and/or cosmeceutical formulation which comprises a composition as described herein in any of the respective embodiments and any combination thereof and, optionally and preferably, a pharmaceutically, cosmetically or cosmeceutically acceptable carrier, preferably a carrier suitable for topical application. The composition as described herein can be used to make-up the formulation by adding the PSO, CBD and CBG as a composition or by adding each of the PSO, the CBD (e.g., isolated CBD) and CBG (e.g., isolated CBG) separately to the formulation.

According to an aspect of some embodiments of the present invention there is provided a cosmetic or cosmeceutical formulation comprising an isolated cannabidiol (CBD), an isolated cannabigerol (CBG) and pomegranate seed oil (PSO).

According to some of any of the embodiments of this aspect of the present invention, a concentration of the PSO in the formulation is no more than 1,000-folds, or no more than 500-folds, or no more than 100-folds, or no more than 50-folds, the concentration of the CBD or CBG in the formulation, as described herein.

According to an aspect of some embodiments of the present invention there is provided a cosmetic or cosmeceutical formulation comprising cannabidiol (CBD), an isolated cannabigerol (CBG) and pomegranate seed oil (PSO), wherein a concentration of the PSO in the composition is no more than 1,000-folds, or no more than 500-folds, or no more than 100-folds, or no more than 50-folds, the concentration of the CBD or CBG.

According to some of any of the embodiments of this aspect of the present invention, the CBD is an isolated CBD, as described herein.

According to some of any of the embodiments of this aspect of the present invention, the CBG is an isolated CBG, as described herein.

According to some of any of the embodiments of these aspects of the present invention, an amount of PSO ranges from 0.01 to 5, or from 0.1 to 4, or from 0.1 to 2, % by weight of the total weight of the formulation, including any intermediate values and subranges therebetween.

According to some of any of the embodiments of these aspects of the present invention, an amount of CBD ranges from 0.1 to 2, % by weight of the total weight of the composition, including any intermediate values and subranges therebetween.

According to some of any of the embodiments of these aspects of the present invention, an amount of CBG ranges from 0.01 to 2, or from 0.01 to 1, % by weight of the total weight of the composition, including any intermediate values and subranges therebetween.

According to some of any of the embodiments of these aspects of the present invention, an amount of PSO ranges from 0.01 to 5, or from 0.1 to 4, or from 0.1 to 2, % by weight of the total weight of the formulation, including any intermediate values and subranges therebetween; an amount of CBD ranges from 0.1 to 2, % by weight of the total weight of the composition, including any intermediate values and subranges therebetween; and an amount of CBG ranges from 0.01 to 2, or from 0.01 to 1, % by weight of the total weight of the composition, including any intermediate values and subranges therebetween.

According to some of any of the embodiments of these aspects of the present invention, an amount of PSO ranges from 0.01 to 5, or from 0.1 to 4, or from 0.1 to 2, % by weight of the total weight of the formulation, including any intermediate values and subranges therebetween; and an amount of CBD ranges from 0.1 to 2, % by weight of the total weight of the composition, including any intermediate values and subranges therebetween.

According to some of any of the embodiments of these aspects of the present invention, an amount of PSO ranges from 0.01 to 5, or from 0.1 to 4, or from 0.1 to 2, % by weight of the total weight of the formulation, including any intermediate values and subranges therebetween; and an amount of CBG ranges from 0.01 to 2, or from 0.01 to 1, % by weight of the total weight of the composition, including any intermediate values and subranges therebetween.

According to some of any of the embodiments of these aspects of the present invention, an amount of CBD ranges from 0.1 to 2, % by weight of the total weight of the composition, including any intermediate values and subranges therebetween; and an amount of CBG ranges from 0.01 to 2, or from 0.01 to 1, % by weight of the total weight of the composition, including any intermediate values and subranges therebetween.

According to some of any of the embodiments of these aspects of the present invention, a weight ratio of CBD to PSO ranges from 2:1 to 1:100, or from 2:1 to 1:50, or from 1:1 to 100:1, including any intermediate values and subranges therebetween.

According to some of any of the embodiments of these aspects of the present invention, a weight ratio of CBG to PSO ranges from 1:5 to 1:100, or from 1:5 to 1:50, including any intermediate values and subranges therebetween.

According to some of any of the embodiments of these aspects of the present invention, a weight ratio of CBD to CBG ranges from 10:1 to 1:1, or from 10:1 to 2:1, including any intermediate values and subranges therebetween.

According to some of any of the embodiments of these aspects of the present invention, a weight ratio of CBD and CBG ranges from 2:1 to 10:1, as described herein, and a weight ratio of PSO and CBD ranges from 1:5 to 50:1, or from 1:2 to 20:1, as described herein.

A formulation according to the present embodiments can be used as a cosmetic or cosmeceutical product (e.g., skin-care product) per se or be used to make up such a product.

In some embodiment of the present invention, the cosmetic or cosmeceutical formulation is formulated in a form suitable for topical application on the applied area (e.g., a keratinous tissue, for example, facial skin).

By selecting the appropriate carrier and optionally other ingredients that can be included in the formulation, as is detailed hereinbelow, the compositions of the present embodiments may be formulated into any form typically employed for topical application.

By “appropriate carrier” for topical application it is meant any medium compatible with a keratinous substrate or a mucosal tissue, which has a color, a smell and a pleasant feel and which does not generate unacceptable discomfort (stinging, tautness or redness).

According to some of any of the embodiments of these aspects of the present invention, the formulation further comprises a cosmetically or cosmeceutically acceptable carrier.

Herein, the term “cosmetically or cosmeceutically acceptable carrier” refers to a carrier or a diluent that does not cause significant irritation to a keratinous material or tissue of an organism and does not abrogate the biological activity and properties of the applied compound or combinations of compounds (e.g., a composition as described herein). Examples, without limitations, of carriers include propylene glycol, water, saline, emulsions and mixtures of organic solvents with water, as well as solid (e.g., powdered) and gaseous carriers. The carrier is typically aimed at facilitating the topical application of the composition to the keratinous tissue or substrate. It is to be noted that the carrier is selected in accordance with the intended use and the form of the formulation or the product containing same and is not limited to the above description.

“Skin-care” means regulating and/or improving a skin condition. Some non-limiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, shiny, and/or dull appearance of skin, improving the hydration status or moisturization of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin-care active” means a compound or combination of compounds that, when applied to skin, provide an acute and/or chronic benefit to skin or a type of cell commonly found therein. Skin-care actives may regulate and/or improve skin or its associated cells (e.g., improve skin elasticity; improve skin hydration; improve skin condition; and improve cell metabolism).

“Skin-care formulation” means a formulation that includes a skin-care active and regulates and/or improves skin condition.

“Skin-care product” as used herein refers to a product that includes a skin-care composition or formulation. Some non-limiting examples of “skin-care products” include skin creams, moisturizers, lotions, and body washes. Other examples are provided hereinbelow

“Topical application” means to apply or spread the formulation, composition or product of the present embodiments onto the surface of the keratinous tissue.

The formulations can be water-based, oil-based, emulsion-based (including water-in-oil, oil-in-water, water-in-oil-in-water and oil-in-water-in-oil emulsions) or silicon-based.

In some embodiments, a formulation as described is in a form of a cream, an ointment, a paste, a gel, a lotion, a milk, an oil, a suspension, a solution, an aerosol, a spray, a foam, a serum, a powder (e.g., a pressed powder or a loose powder) or a mousse.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emolliency). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the sunscreens-containing capsules, are present in a water or alcohol base. Lotions are typically preferred for covering/protecting large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol and/or natural oil substances and/or plant extracts. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base.

Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the skin of a user, quickly evaporates, driving the active ingredient through the upper skin layers to the site of treatment.

In any of the formulations or products described herein, additional agents and/or additives can be included.

Some non-limiting representative examples of additives and/or agents include humectants, deodorants, antiperspirants, sunscreen agents (e.g., UV blocking agents, UV filters), sunless tanning agents, hair conditioning agents, pH adjusting agents, chelating agents, preservatives, emulsifiers, occlusive agents, emollients, thickeners, solubilizing agents, penetration enhancers, anti-irritants, colorants, propellants and surfactants.

Representative examples of humectants include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propyleneglycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Suitable pH adjusting agents include, for example, one or more of adipic acids, glycines, citric acids, calcium hydroxides, magnesium aluminometasilicates, buffers or any combinations thereof.

Representative examples of deodorant agents include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmithyl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate.

Other deodorant agents include, without limitation, odor absorbing materials such as carbonate and bicarbonate salts, e.g. as the alkali metal carbonates and bicarbonates, ammonium and tetraalkylammonium carbonates and bicarbonates, especially the sodium and potassium salts, or any combination of the above.

Antiperspirant agents can be incorporated in the compositions of the present invention either in a solubilized or a particulate form and include, for example, aluminum or zirconium astringent salts or complexes.

Representative examples of sunless tanning agents include, without limitation, dihydroxyacetone, glyceraldehyde, indoles and their derivatives. The sunless tanning agents can be used in combination with the sunscreen agents.

The chelating agents are optionally added to formulations so as to enhance the preservative or preservative system. Preferred chelating agents are mild agents, such as, for example, ethylenediaminetetraacetic acid (EDTA), EDTA derivatives, or any combination thereof.

Suitable preservatives include, without limitation, one or more alkanols, disodium EDTA (ethylenediamine tetraacetate), EDTA salts, EDTA fatty acid conjugates, isothiazolinone, parabens such as methylparaben and propylparaben, propyleneglycols, sorbates, urea derivatives such as diazolindinyl urea, or any combinations thereof.

Suitable emulsifiers include, for example, one or more sorbitans, alkoxylated fatty alcohols, alkylpolyglycosides, soaps, alkyl sulfates, monoalkyl and dialkyl phosphates, alkyl sulphonates, acyl isothionates, or any combinations thereof.

Suitable occlusive agents include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients include, for example, dodecane, squalane, cholesterol, isohexadecane, isononyl isononanoate, PPG Ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids and their alkali salts and mixtures thereof.

Representative examples of solubilizing agents that are usable in this context of the present invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEENS and spans, e.g., TWEEN 80. Other solubilizers that are usable for the compositions of the present invention are, for example, polyoxyethylene sorbitan fatty acid ester, polyoxyethylene n-alkyl ethers, n-alkyl amine n-oxides, poloxamers, organic solvents, phospholipids and cyclodextrines.

Suitable penetration enhancers include, but are not limited to, dimethylsulfoxide (DMSO), dimethyl formamide (DMF), allantoin, urazole, N,N-dimethylacetamide (DMA), decylmethylsulfoxide (Cio MSO), polyethylene glycol monolaurate (PEGML), propyleneglycol (PG), propyleneglycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, and the like. The permeation enhancer may also be a vegetable oil. Such oils include, for example, safflower oil, cottonseed oil and corn oil.

Suitable anti-irritants include, for example, steroidal and non-steroidal anti-inflammatory agents or other materials such as aloe vera, chamomile, alpha-bisabolol, cola nitida extract, green tea extract, tea tree oil, licoric extract, allantoin, caffeine or other xanthines, glycyrrhizic acid and its derivatives.

Exemplary additional active agents according to some embodiments of present invention include, without limitation, one or more, or any combination of an anti-acne agent, an anti-aging agent, a wrinkle-reducing agent, a skin whitening agent, a sebum reducing agent, an anesthetic agent, an antipruriginous agent, an antineoplastic agent, an immunomodulator, an interferon, an antidepressant, an anti-histamine, a vitamin, a mineral (e.g., a dead sea mineral), a hormone and an anti-dandruff agent.

Exemplary additional active agents according to some embodiments of present invention include, without limitation, one or more, or any combination of a vitamin, a mineral (e.g., a dead sea mineral), a ceramide, hyaluronic acid, a protein (e.g., collagen, elastin), a hydroxy acid (e.g., alpha or beta hydroxyacid), a peptide, an amino acid, a sunscreen agent, a herb or plant extract, a fungus, a herb (e.g., a medicinal herb, a seaweed, an alga, and more. In exemplary embodiments, and depending on the intended use of the formulation and its form, an amount of the composition in the formulation ranges from 0.1 to about 10% by weight of the total weight of the formulation, including any intermediate values and subranges therebetween.

According to some embodiments of the present invention, the formulation or product is devoid of an anti-inflammatory agent and/or an anti-microbial agent and/or an antioxidant, other than CBD, CBG and PSO.

According to some embodiments of the present invention, the formulation or product is devoid of an anti-microbial agent other than the CBD, CBG and PSO.

According to some of any of the embodiments described herein, the composition or the formulation is devoid of a cannabinoid other than CBD and CBG as described herein.

According to some of any of the embodiments described herein, the composition or the formulation is devoid of a material extracted from a cannabis plant other than CBD and CBG as described herein.

By “devoid of” it is meant that the composition, formulation or product includes no more than 0.5%, or no more than 0.1%, or no more than 0.05%, or no more than 0.01%, or no more than 0.001%, or null, of the indicated substance.

According to some of any of the embodiments described herein, at least some (e.g., at least 20%, or at least 30%, or at least 40%, or at least 50%, or at least 60%, or at least 70%, or at least 80%), and preferably all of the components that are present in the formulation or product in addition to the PSO, CBD and CBG, are natural substances, that is, substances that are present, derived from or extracted from, natural sources such as plants or minerals.

Exemplary natural substances include, but are not limited to, water, glycerin, natural gums such as xanthan gum, caesalpinia spinose gum, glyceryl esters, laurates, behenyl alcohol, shea butter, mango butter, lavender butter, squalane, bisabolol, D. panthenol, orange oil, bergamot oil, substances such as marketed under the trade name “Lanette O”, “SPAN 80”, “Eutanol G”, “Emogreen C69”, “Actifoil Spotless Zro”, tocopherol acetate, cocglycerides, octyl palmitate, tocopherol, phenthylene glycol, soya oil, raspberry seed oil, cucumber oil, sunflower oil, niacinamide, plant extracts such as ginseng extract, lavender extract, natural nanogen, ethylhexylglycerin, coccaprylate, ascorbyl tetraisopalmitate, octyldodecanol, and any other natural substance that is functionally equivalent to any of the foregoing.

Exemplary formulations are described in the Examples section that follows. According to some of any of the embodiments of these aspects of the present invention, an exemplary formulation in a form of an emulsion (e,g., a cream) comprises PSO in an amount of from 0.1 to 1, or of 0.2 to 8, or from 0.2 to 0.6, for example about 0.5%, by weight; CBD in an amount of from 0.1 to 2, or from 0.5 to 1.5, or from 0.8 to 1, for example, from 0.9 to 1, % by weight; and CBG in an amount of from 0.01 to 1, or from 0.02 to 0.08, or about 0.05, % by weight. Such formulations typically further include an aqueous phase in an amount of at least 50% by weight, and an oily phase that comprises oily substances, typically natural oily substances and plant extracts.

According to some of any of the embodiments of these aspects of the present invention, an exemplary formulation in a form of an oil, such as, a cleansing oil, comprises PSO in an amount of from 1 to 10, or of 1 to 8, or from 1 to 6, or from 2 to 6, for example 4, % by weight; CBD in an amount of from 0.1 to 1, or from 0.1 to 0.5, for example, from 0.2 to 0.4, % by weight; and CBG in an amount of from 0.01 to 1, or from 0.05 to 0.5, or about 0.1, % by weight. Such formulations typically further include oily substances, typically natural oily substances and plant extracts.

According to some of any of the embodiments of these aspects of the present invention, an exemplary formulation in a form of concentrated oil, such as a serum, comprises PSO in an amount of from 1 to 10, or of 1 to 5, or from 1 to 3, for example 2, % by weight; CBD in an amount of from 0.1 to 10, or from 1 to 10, or from 1 to 5, or from 1 to 3, for example, from 1 to 2, % by weight; and CBG in an amount of from 0.01 to 1, or from 0.01 to 0.1, or from 0.05 to 0.1, % by weight. Such formulations typically further include oily substances, typically natural oily substances and plant extracts.

The cosmetic or cosmeceutical formulations as described herein in any of the respective embodiments and any combination thereof can be packaged in a packaging material and identified in print, in or on the packaging material, for use in the treatment of a medical or cosmetic condition, as described in further detail hereinbelow. The package can be adopted for topical application of the formulation, in accordance with its consistency. For example, liquid formulations (including emulsions and oils) can be packaged in a container having means to topically apply the formulation, for example, a nozzle, a dropper, a pipette, or in a narrow-necked and/or squeezable bottle.

Formulations in a form of a spray or aerosol can be packaged in a container equipped with a spraying nozzle and/or mechanism. Ointments, gels, oils and creams can be packaged in a simple container, or in a squeezable container.

According to an aspect of some embodiments of the present invention there is provided an article-of-manufacturing, which comprises any of the formulations and/or products as described herein, and optionally means for topically applying the formulations (e.g., as described herein).

Uses:

The formulations as described herein can be or be used to make up a product, for example, skin care products, make-up products (including eye shadows, make-up, lipstick, lacquer, etc.), men's grooming products, sunscreen products, hair care products, or any other product as described herein.

Exemplary products include, but are not limited to, a cleansing product (e.g., body and/or face and/or hygienic soap, shampoo, conditioner), and anti-wrinkle product, an anti-ageing product, a sebum reducing product, a product with anesthetic activity, a mouth wash product, a sunscreen product, and any other product that can benefit from the anti-inflammatory, anti-bacterial and/or anti-oxidation properties of the composition.

Exemplary products include, but are not limited to, an anti-acne product, an anti-diaper rush product, a shampoo, a body lotion, a body cream, a face cream, a face lotion, a face mask, a body and/or face wash product, a cleansing product (for hair and/or skin and/or mucosal tissue such as oral cavity or vagina), a hygienic product, an anti-pigmentation product, a make-up product, an anesthetic product, a sunscreen product.

Exemplary skin care products include products for improving skin appearance and/or feel by providing a smoother, more even appearance and/or feel; products for increasing the thickness of one or more layers of the skin; products for improving the elasticity or resiliency of the skin; products for improving the firmness of the skin; products for reducing the oily, shiny, and/or dull appearance of skin, products for improving the hydration status or moisturization of the skin, products for improving the appearance of fine lines and/or wrinkles, products for improving skin exfoliation or desquamation, products for plumping the skin, products for improving skin barrier properties, products for improving skin tone, products for reducing the appearance of redness or skin blotches, and products for improving the brightness, radiancy, or translucency of skin. Such products are to be applied, for example, to a facial skin, the neck, the torso, and/or the décolleté.

The formulations and products as described herein can be used to treat, prevent (protect from) or reduce damage to a keratinous and/or mucosal tissue of a subject.

The formulations and products as described herein can be used to treat, prevent or ameliorate a medical and/or cosmetic condition in which topical administration of a composition that exhibits anti-inflammatory and anti-oxidative activities is beneficial.

Medical, cosmetic or cosmeceutical conditions that can benefit from topical application of the compositions, formulations or products as described herein include, but are not limited to, damage to skin cells caused by UV radiation, skin aging, skin pigmentation, stress, extreme weather conditions, menopause, xerosis, ichthyosis, keratosis, keratoderma, pruritus, acne, dermatitis, neuro-dermatitis, dermatitis herpetiformis, actinic keratosis, hyper keratosis, inflamed keratosis, eczema, atopic eczema, melanoma, psoriasis, rosacea, urticaria, seborrheic dermatitis, skin cancer, xeroderma pigmentosum, infections caused by pathogenic microorganisms, wounds, inflammation and/or pain, inflammatory diseases or disorders such as tinea pedis (athlete's foot), acne, tinea versicolor (a benign scaly skin condition), and thrombophlebitis, primary Raynaud's phenomenon (PRP), limited cutaneous systemic sclerosis (LCSSc), diabetic ulcer wounds, skin infections, eczema, rash, immunologically mediated systemic diseases, allergic response-mediated systemic diseases, viral blisters such as one caused by herpes, anal fissure, anal fissure pain, post Hirschprung surgery (for the treatment of obstructive symptoms), acute strangulated internal hemorrhoids, pain and symptoms of chronic extensor tendinosis of the elbow (tennis elbow).

According to some embodiments, the keratinous tissue is a facial tissue, a hair tissue, or a skin tissue (e.g., of the décolleté).

According to some embodiments, the mucosal tissue is a vaginal tissue.

According to some embodiments, the mucosal tissue is in the oral cavity.

According to some embodiments, the damage is associated with aging.

According to some embodiments, the damage is associated with a dermatological condition, for example, skin inflammation, and/or conditions such as atopic dermatitis, psoriasis, pigmentation, acne, eczema, xerosis, ichthyosis, keratosis, keratoderma, pruritus, dermatitis, neuro-dermatitis, dermatitis herpetiformis, actinic keratosis, hyper and inflamed keratosis, atopic eczema, melanoma, rosacea, urticaria, seborrheic dermatitis, skin cancer, and xeroderma pigmentosum.

According to some embodiments, any of the compositions, formulations, articles-of-manufacturing and/or products as described herein, is packaged in packaging material and identified in print, in or on the packaging material, for its intended use.

The compositions as described herein can be identified for use in preparing any of the formulations or products or articles-of-manufacturing as described herein.

The compositions as described herein can be identified for use in preparing any of the products or articles-of-manufacturing as described herein.

The products and/or articles-of-manufacturing as described herein can be identified for an intended use, as described herein in any of the respective embodiments, depending on the formulation that makes up the product or article, and on other ingredients that may be included in the product.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Samples

Materials:

Cannabidiol (CBD) and Cannabigerol (CBG), as isolates, or isolated samples, in a solid form (e.g., powder) were obtained from Cannabis Sativa L.. Briefly, part of the plant (mainly leaves and infructescence; 2 mg) were dissolved in 1 ml of a diluent (80% methanol) and 2 microliter (μL) were directly injected to ultra-performance liquid chromatographer (UPLC).

Pomegranate Seed Oil (PSO) was obtained from a commercial vendor.

Tested Samples:

Isolated samples of CBD and CBG, PSO and combination samples thereof were used.

Stock solutions of isolated CBD and CBG were prepared by dissolving the isolates.

Pomegranate seed oil (PSO) solutions were prepared in two steps. First PSO was dissolved in ethanol at a ratio up to 2:1 and then cell media was added in order to make a desired final concentration. The calculations were based on the PSO weight (10 μl oil=8 mg).

All the stock solutions were freshly prepared before each experiment and their concentrations are presented below.

Stock solutions of CBD and of CBG were prepared according the desired final concentration in each experiment. For example, a 10 μM stock solution of CBG was prepared by dissolving 1.42 μg of the isolate in 448 μl of EtOH; a 50 μM stock solution of CBG was prepared by dissolving 1.4 μg of the isolate in 88.47 μl of EtOH. A 10 μM stock solution of CBD was prepared by dissolving 1.26 μg of the isolate in 400 μl of EtOH; a 40 μM stock solution of CBD was prepared by dissolving 1.45 μg of the isolate in 115.27 μl of EtOH.

A stock solution of 40 μg/μl PSO was prepared by dissolving 20 μl of the oil in 10 μl of EtOH and then in 397 μl of the medium used in the respective assay. Stock solutions of CBD, CBG and PSO were prepared by adding a stock solution of CBD and a stock solution of CBG to the stock solution of PSO (in ethanol and medium) so as to achieve the desired final concentration of each material (see, Tables 2A-B).

Table 1 below presents exemplary combination samples used in the following studies and the concentration ratio therebetween.

Tables 2A (for sample combinations 1-5) and 2B (for sample combinations 6-10) below present schematically how samples featuring the indicated concentrations of each component were obtained.

100 μL of a stock solution of the combination samples were added to the cells. For the isolated samples, the stock solutions were mixed in the medium for achieving the desired final concentration.

Table 3 below resents the absolute weight of each of the components in each combination sample and the weight ratio therebetween.

TABLE 1

| Comb. | CBD | CBD | CBG | CBG | PSO | |
|---|---|---|---|---|---|---|
| No. | (μM) | (μg/μl) | (μM) | (μg/μl) | (μg/μl) | Ratio |
| 1 | 20 | about | 40 | about | 40 | 1:2:about |
| | | 0.063 | | 0.127 | | 600 |
| 2 | 20 | about | 40 | about | 5 | 1:2:about |
| | | 0.063 | | 0.127 | | 80 |
| 3 | 10 | about | 40 | about | 5 | 1:4:about |
| | | 0.03145 | | 0.127 | | 160 |
| 4 | 10 | about | 10 | about | 40 | 1:1:about |
| | | 0.03145 | | 0.03165 | | 600 |
| 5 | 5 | about | 5 | about | 5 | 1:1:about |
| | | 0.0157 | | 0.016 | | 300 |
| 6 | 10 | about | 5 | about | 0.01 | 2:1:1 |
| | | 0.03145 | | 0.016 | | |
| 7 | 10 | about | 20 | about | 2.5 | 1:2:about |
| | | 0.03145 | | 0.063 | | 80 |
| 8 | 10 | about | 20 | about | 0.01 | 3:6:1 |
| | | 0.03145 | | 0.063 | | |
| 9 | 10 | about | 10 | about | 0.01 | 3:3:1 |
| | | 0.03145 | | 0.03165 | | |
| 10 | 5 | about | 5 | about | 0.005 | 3:3:1 |
| | | 0.0157 | | 0.016 | | |

TABLE 2A

| | Stock | Amount of | Stock | Amount of | | | |
|---|---|---|---|---|---|---|---|
| Combo | CBD | Stock CBD | CBG | Stock CBG | PSO | Ethanol | Medium |
| No. | (μM) | (μL) | (μM) | (μL) | (μL) | (μL) | (μL) |
| 1 | 40 | 2 | 50 | 3.2 | 20 | 10 | 364.8 |
| 2 | 40 | 2 | 50 | 3.2 | 2.5 | 10 | 382.3 |
| 3 | 20 | 2 | 50 | 3.2 | 2.5 | 10 | 382.3 |
| 4 | 20 | 2 | 12.5 | 3.2 | 20 | 10 | 364.8 |
| 5 | 20 | 2 | 12.5 | 3.2 | 2.5 | 10 | 382.3 |

TABLE 2B

| | Stock | Amount of | Stock | Amount of | Stock | Amount | |
|---|---|---|---|---|---|---|---|
| Combo | CBD | Stock CBD | CBG | Stock CBG | PSO | of Stock | Medium |
| No. | (μM) | (μL) | (μM) | (μL) | (μg/μL) | PSO (μL) | (μL) |
| 6 | 1 | 5 | 1 | 2.5 | 1 | 5 | Balance |
| | | | | | | | to 500 |
| 7 | 1 | 5 | 1 | 10 | 40 | 31.25 | Balance |
| | | | | | | | to 500 |
| 8 | 1 | 5 | 1 | 10 | 1 | 5 | Balance |
| | | | | | | | to 500 |
| 9 | 1 | 5 | 1 | 5 | 1 | 5 | Balance |
| | | | | | | | to 500 |
| 10 | 1 | 2.5 | 1 | 2.5 | 1 | 2.5 | Balance |
| | | | | | | | to 500 |

TABLE 3

| Combo | CBD | CBD | CBG | CBG | PSO | PSO | |
|---|---|---|---|---|---|---|---|
| No. | (mg) | (% wt.) | (mg) | (% wt.) | (mg) | (% wt.) | Ratio |
| 1 | 25.16 | 27 | 50.64 | 55 | 16 | 17.4 | 2:3:1 |
| 2 | 25.16 | 32 | 50.64 | 63 | 4 | 5 | 6:12:1 |
| 3 | 12.6 | 19 | 50.64 | 76 | 4 | 6 | 1:3:3 |
| 4 | 12.6 | 30 | 12.6 | 30 | 16 | 40 | 1:1:1.3 |
| 5 | 12.6 | 43 | 12.6 | 43 | 4 | 14 | 3:3:1 |
| 6 | 1.6 | 66 | 0.8 | 33 | 0.005 | 0.2 | 320:160:1 |
| 7 | 1.6 | 27 | 3.2 | 53 | 1.25 | 20 | 1.3:2.6:1 |
| 8 | 1.6 | 33 | 3.2 | 66 | 0.005 | 0.1 | 320:640:1 |
| 9 | 1.6 | 50 | 1.6 | 50 | 0.005 | 0.15 | 320:320:1 |
| 10 | 0.8 | 50 | 0.8 | 50 | 0.0025 | 0.15 | 32:32:1 |

### Example 2

### Activity Assays

Materials and Experimental Methods

Cells Culture and Treatments:

Cell lines were cultured according to standard mammalian tissue culture protocols and sterile technique. Human monocytes THP-1 cells were cultured in RPMI medium 1640. HaCaT cells were cultured in DMEME medium. All media was supplemented with 10% fetal bovine serum, streptomycin (100 mg/ml), penicillin (100 U/ml) and Nystatin (12.5 U/ml). Cells were incubated in 5% CO₂ at 37° C. All tissue culture cells were maintained in 75 cm² cell culture treated flask (Eppendorf) and all the media and supplements were obtained from Biological Industries. Treatment were performed by plating cells in a 96 micro well delta surface plates (Eppendorf) in a starting confluence of 1×104 cells/well.

Determination of Cells Viability Using MTT Assay:

The viability of the cells following treatment was determined using a commercially available MTT assay kit (Abcam, ab146345) and performed according to manufacturer's instructions. Briefly, cells were seeded in a 96-well plate at a density of 1×104 cells/well (n=4) in 100 μl cell specific media. After overnight incubation, cells were exposed to varying concentrations of isolated components (CBD, CBG and PSO, each alone) and combination samples as described in Example 1, in 100 μl of specific media, by adding a stock solution to the medium in amount that provides the indicated concentration (see, Tables 1, 2A and 2B). Then, plates were incubated in a humidified atmosphere containing 5% CO₂ in air at 37° C. for 24 hours. According to the MTT standard protocol, after 24 hours treatment, the media was removed, and all cells were incubated with serum-free media containing 0.5 mg/ml MTT for 4 hours at the incubator. The MTT purple crystals formed by the viable cells were dissolved using isopropanol containing 0.04 mol/L HCl. The quantification was determined by measuring the optical density at 570 nm in a spectrophotometer reader (Spark, Tecan). Results are presented as proportional viability (%) by comparing between treated and untreated groups.

Cytotoxic potential of an extract was determined according to the following criteria:

Cytotoxic: Viability is reduced to less than 70% of vehicle control.

Non-cytotoxic: Viability is >70% of vehicle control

Determination of cellular ROS (Reactive Oxygen Species) production:

Cellular ROS production was measured using a DCFDA assay kit (Abcam, ab113851)) according to the manufacturer's protocol. DCFDA (2′,7′-dichlorofluorescein diacetate), a cell-permeable fluorogenic dye, is deacetylated to a non-fluorescent compound by cellular esterases and later oxidized into highly fluorescent DCF (2′,7′-dichlorofluorescein) by ROS, allowing measurement of hydroxyl, peroxyl and other ROS activity within the cell.

HaCaT cells were seeded at a density of 2.5×10⁴ for 24 hours on 96-well plates. After 24 hours, the cells were washed twice with 1× buffer assay and then incubated with 25 μM DCFDA in 1× buffer assay in a 37° C. incubator for 45 minutes. After two washes with 1× buffer assay, the cells were exposed to the tested samples and to 0.25 M H202, each added separately to the medium.

Cell fluorescence in the 96-well plates was measured at 488 nm excitation and 525 nm emission using a Tecan spark plate reader (Tecan US, Inc. Morrisville, NC), 5 hours after the addition of the tested samples or sample combinations.

The control used as reference are cells treated only with H₂O₂. The results are shown as percentage of the control.

Detection of cytokine secretion using Homogeneous Time-Resolved fluorescence (HTRF):

Human THP-1 monocytes were seeded in a 96-well plate (Eppendorf) at a density of 3×105 cells/well (n=4) in 100 μl cell specific media and was differentiated using phorbol-12-myristate 13-acetate (PMA) (Sigma-Aldrich) at a final concentration of 200 nM. PMA supplemented media was removed after 72 hours. Cells were then washed twice with PBS and rested in fresh PMA-free media for further 24 hours in order to obtain phenotypic characteristics of macrophages (Daigneault et al., 2010, PLoS One, 5, e8668).

After 24 hours, cells were stimulated by Lipopolysaccharides (LPS) at a final concentration of 1μg/ml and were treated with the samples using specific media containing 5% FBS (100 μL of media solution containing the tested materials at the indicated concentration; see, Example 1).

After 24 hours, the cells supernatants were harvested and diluted up to 5 times in working media to avoid the hook effect. HTRF assays were performed in white 96-well plate (CisBio Bioassays) with a total working volume of 20 μL. All HTRF reagents were purchased from CisBio Bioassays and reconstituted according to the supplier protocols. For each assay 16 μL of diluted supernatants samples were incubated with 4 μL mixed solution containing donor and acceptor antibodies. Antibodies for the detection of TNFα and IL6 were used.

After 2 hours incubation, HTRF signals were measured using SPARK® multimode microplate reader with an excitation filter at 320 nm and fluorescence wavelength measurement at 620 and 665 nm, an integration delay of 60 μs and an integration time of 400 μs. Results were analyzed with a two-wavelength signal ratio: [intensity (665 nm)/intensity (620 nm)]*104. Calculation and analysis of cytokines release was performed according to the supplier protocols and using the 4PL 1/Y2 formula in GraphPad Prism software.

Results:

Effect on viability of THP-1 Cells:

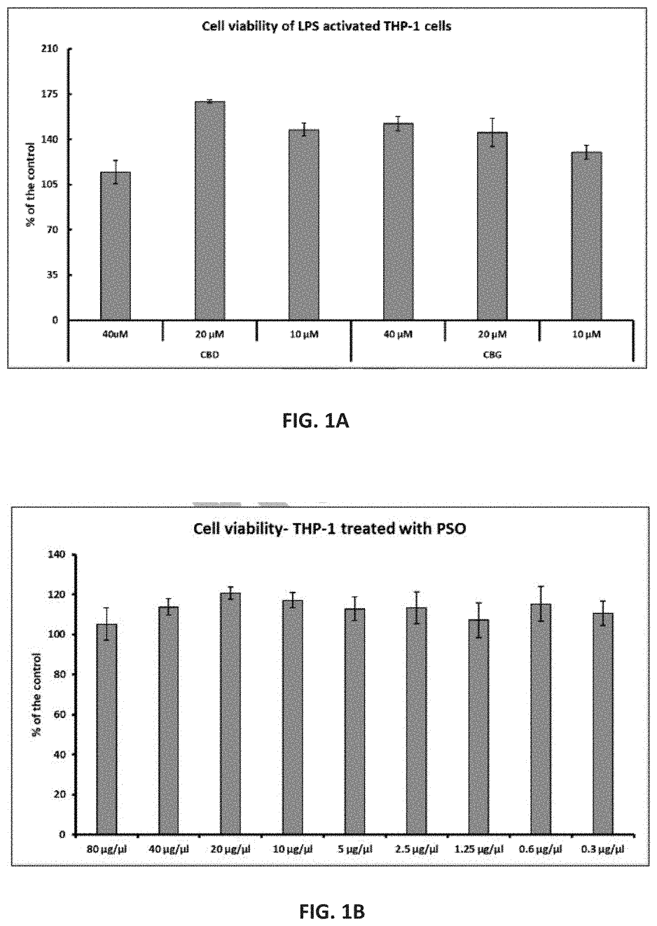

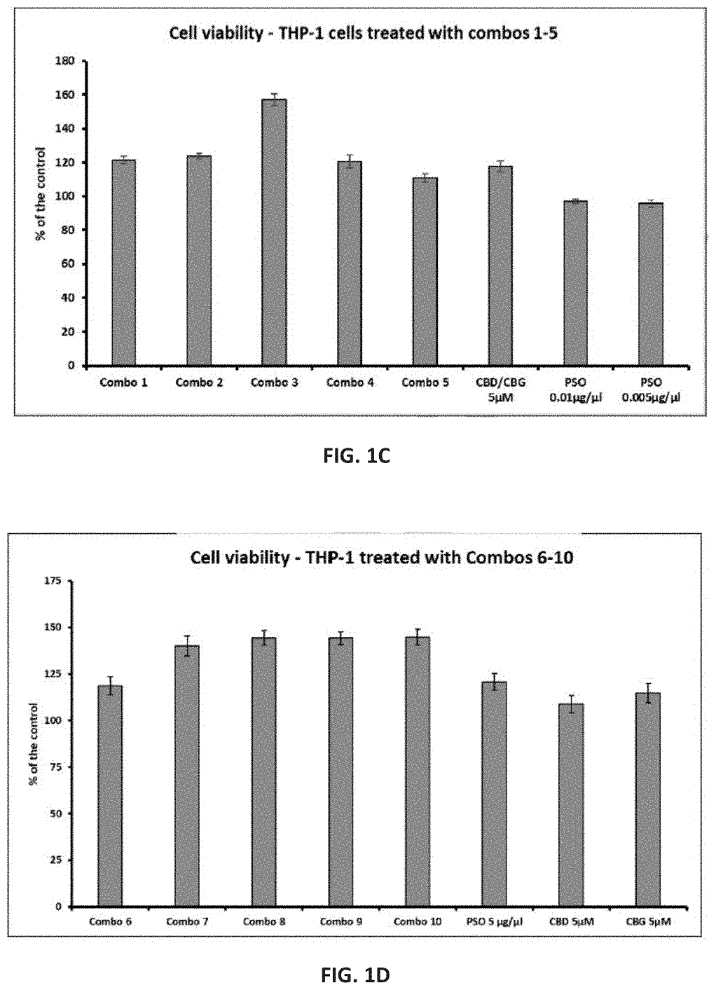

FIGS. 1A-B present the effect of various concentrations of CBD or CBG (FIG. 1A) and of PSO (FIG. 1B) on THP-1 cells viability. The effect of the tested combination samples 1-10 (see, Table 1 above) on THP-1 cell viability is shown in FIG. 1C for comb. Samples 1-5, and in FIG. 1D, for comb. Samples 6-10.

As can be seen in FIG. 1A, CBD and CBG, at all tested concentrations, resulted in enhanced viability, with the most pronounced effect exhibited for CBD at 20 μM.

As can be seen in FIG. 1B, PSO exhibited the same effect on THP-1 viability at all tested concentrations.

As can be seen in FIGS. 1C-D, combination sample 3 and all of combination samples 6-10 exhibited the higher effect on the viability of THP-1 cells. Effect on Cytokine Secretion from LPS-activated THP-1 Cells:

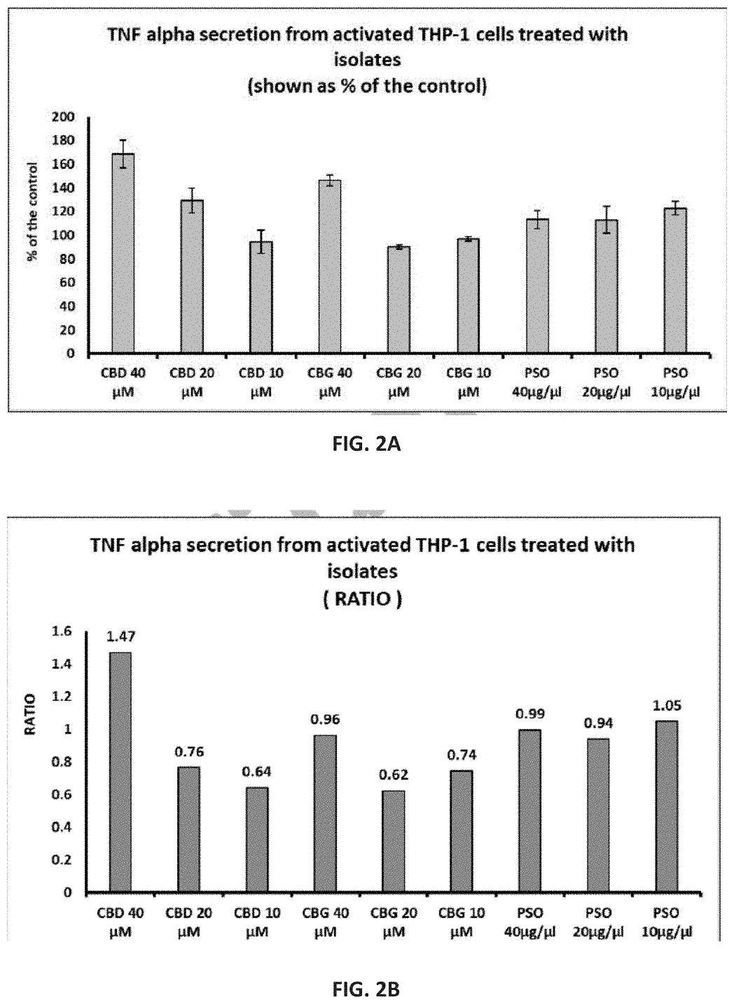

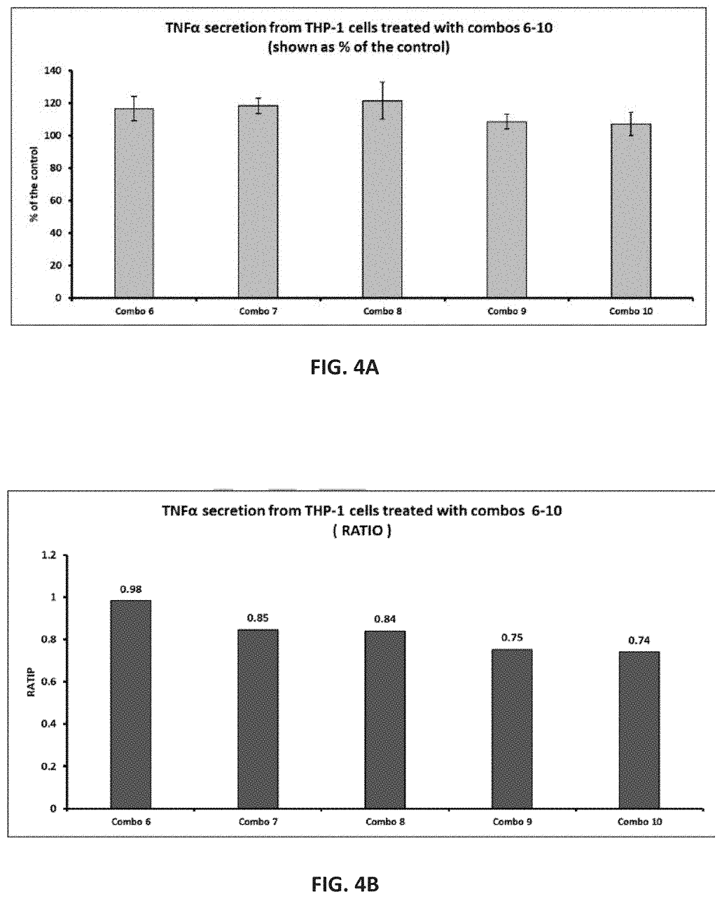

FIGS. 2A-B, 3A-B and 4A-B present the effect of various concentrations of CBD, CBG or PSO (each alone) and of combination samples 1-10 (see, Table 1) on TNFα secretion from LPS-stimulated THP-1 cells. The results are shown as percentage of the control for FIG. 2A, 3A and 4A, and as the ratio between the cells' viability values and the TNFα secretion values, for a same given well (FIGS. 2B, 3B and 4B).

As can be seen in FIGS. 2A-B, treatment with CBD and CBG at the highest concentration resulted in elevation of TNFα secretion, and CBD at the lowest concentration and CBG at a medium concentration resulted in the lowest ratio of TNFα secretion to cells viability.

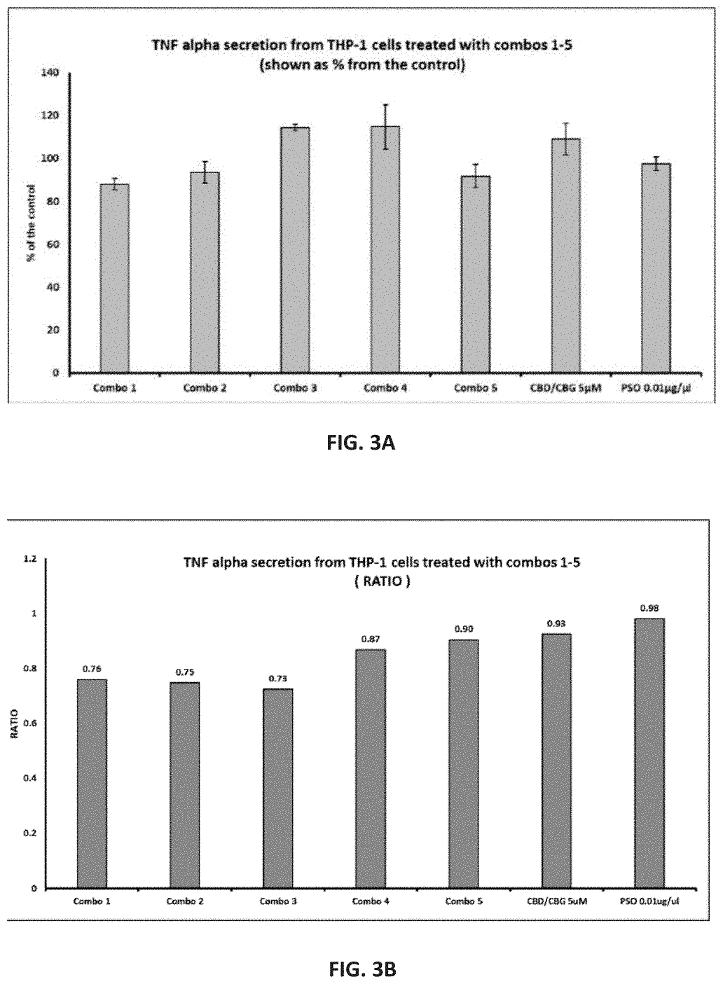

As can be seen in FIGS. 3A-B and 4A-B, of all the tested combination samples, treatment with combination sample 1 resulted in the lowest level of TNFα secretion, and combination samples 1-3 and 6-10 resulted in the lowest ratios of TNFα secretion to cells viability.

These data show that CBD at 10 μM and 20 μM and CBG at 20 μM exerted a mild effect (ratio of about 0.6) in reducing the TNFα secretion levels, and acted as high pro-proliferation agents (140% cells viability). Sample combinations 1, 2, 3, 9 and 10 also produced mild effect (ratio of about 0.75) in reducing TNFα secretion levels and high viability values (above 120%).

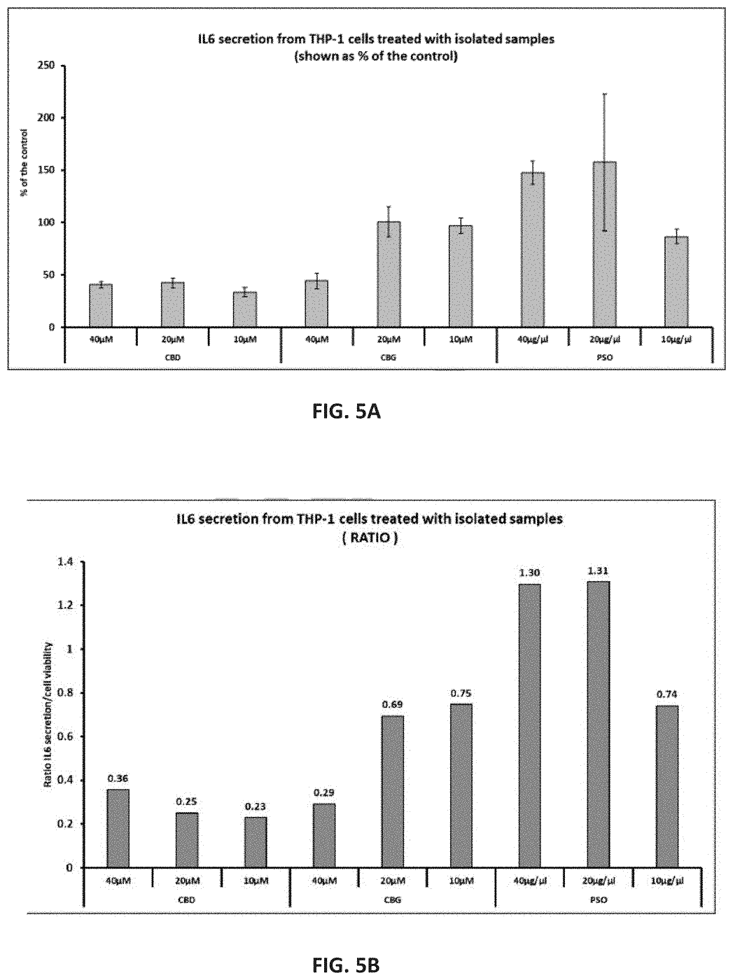

FIGS. 5A-B, 6A-B and 7A-B present the effect of various concentrations of CBD, CBG or PSO (each alone) and of combination samples 1-10 (see, Table 1) on IL-6 secretion from LPS-stimulated THP-1 cells. The results are shown as percentage of the control for FIGS. 5A, 6A and 7A, and as the ratio between the cells' viability values and the TNFα secretion values, for a same given well (FIGS. 5B, 6B and 7B).

As can be seen in FIGS. 5A-B, CBD and CBG at the highest concentration reduced IL-6 secretion by more than 50%. CBG at the same concentration also showed high reduction of the ratio between IL-6 secretion and viability. PSO at high concentration resulted in elevated level of IL-6 secretion.

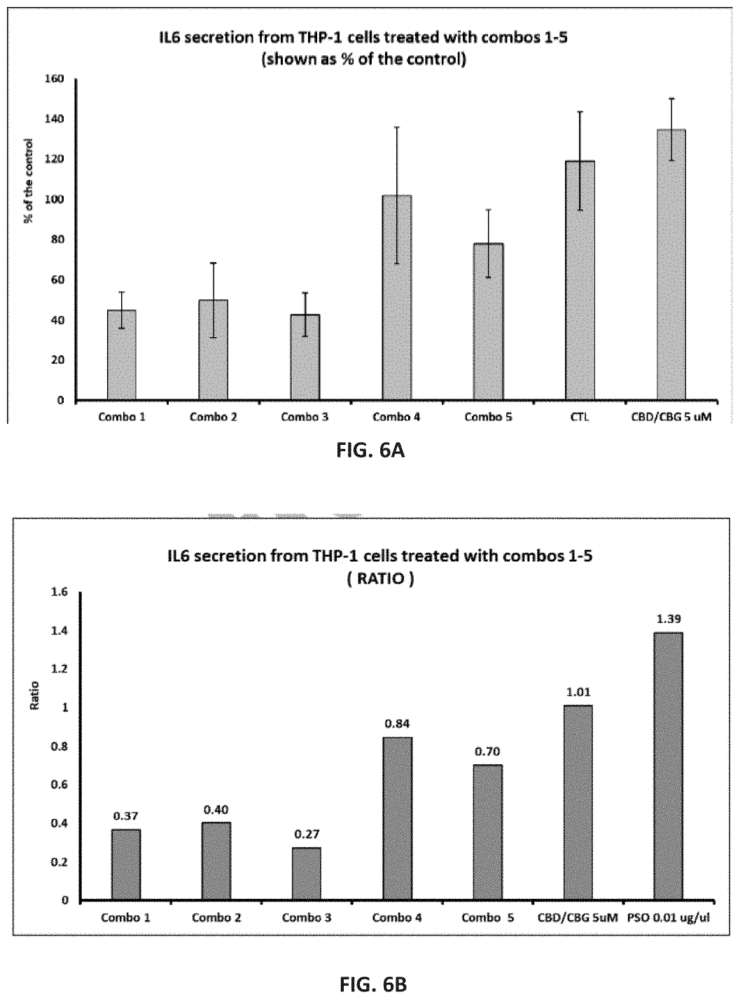

As can be seen in FIGS. 6A-B, sample combinations 1-3 showed reduction of more than 50% in IL-6 secretion and a lowest reduction in the ratio to cells viability.

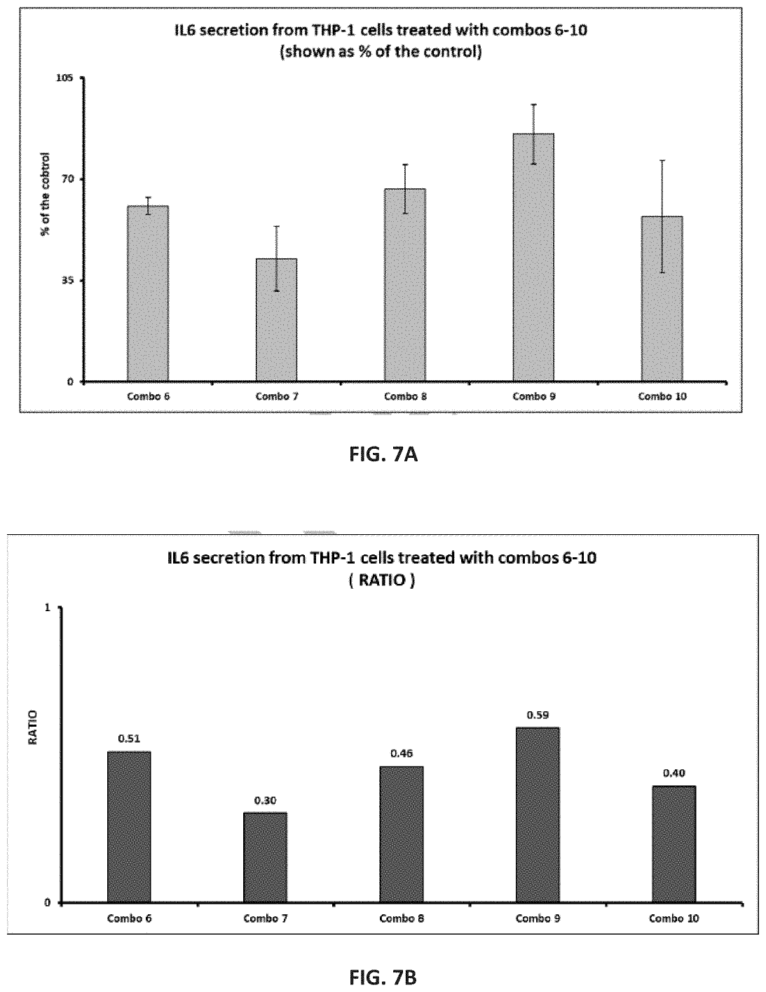

As can be seen in FIGS. 7A-B, all sample combinations 6-10 showed reduction in IL-6 secretion and in the ratio to cells viability, with sample combinations 7 and 10 exhibiting higher effects.

These data show that CBD produced a very strong effect in reducing IL6 secretion levels from activated THP-1 cells including at the lowest tested concentration of 10 μμM (ratio of 0.23). CBG at 40 μM was also highly potent (ratio of 0.29). These treatments also resulted in very high viability levels (above 140% for CBD and CBG). Sample combinations 1, 2, 3, 7 and 10 produced high effect in reducing IL6 levels (ratio of 0.27 to 0.4), and mostly also resulted in high viability levels for some of the combos.

Effect on ROS Production from HaCaT Cells:

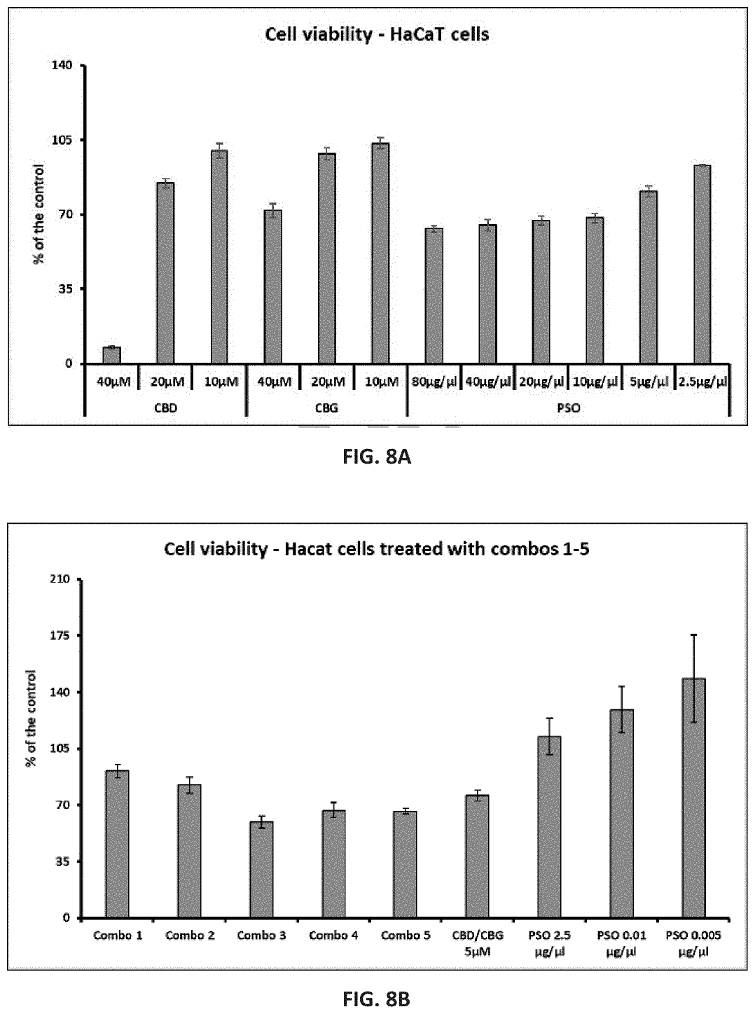

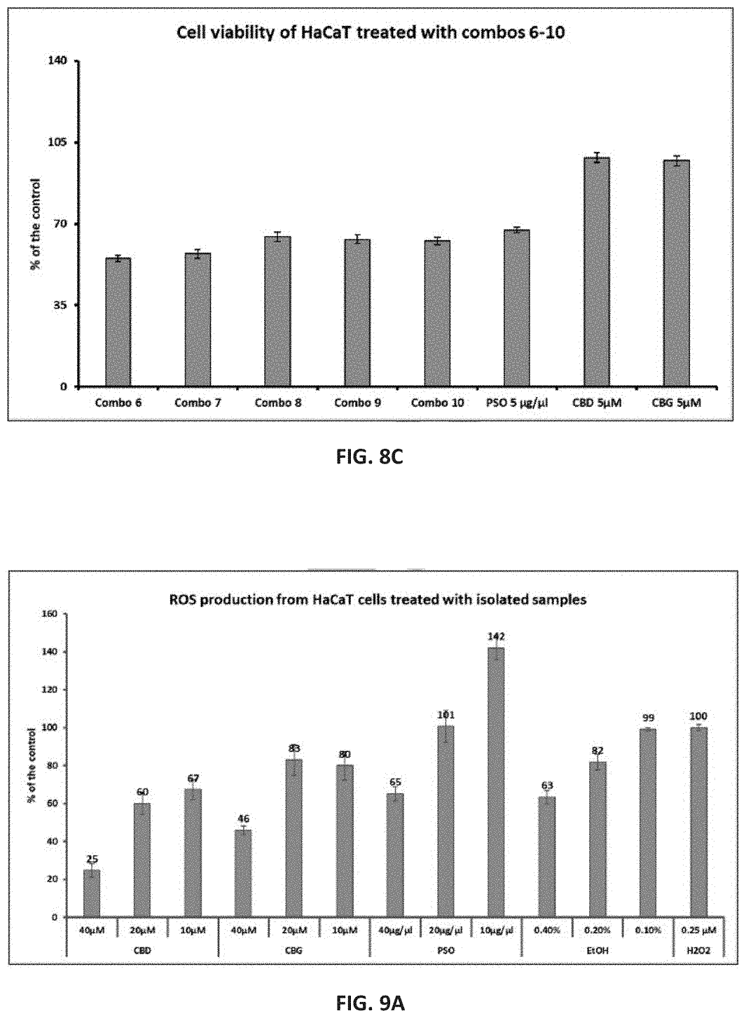

FIGS. 8A-C show the effect of various concentrations of CBD, CBG or PSO (FIG. 8A), of sample combinations 1-5 (FIG. 8B) and of sample combinations 6-10 (FIG. 8C) on the viability of HaCaT cells.

As can be seen, CBD at the higher concentration was found to be toxic. Samples combinations 3-5 and 6-10 exhibited an effect that is below toxic level (70%).

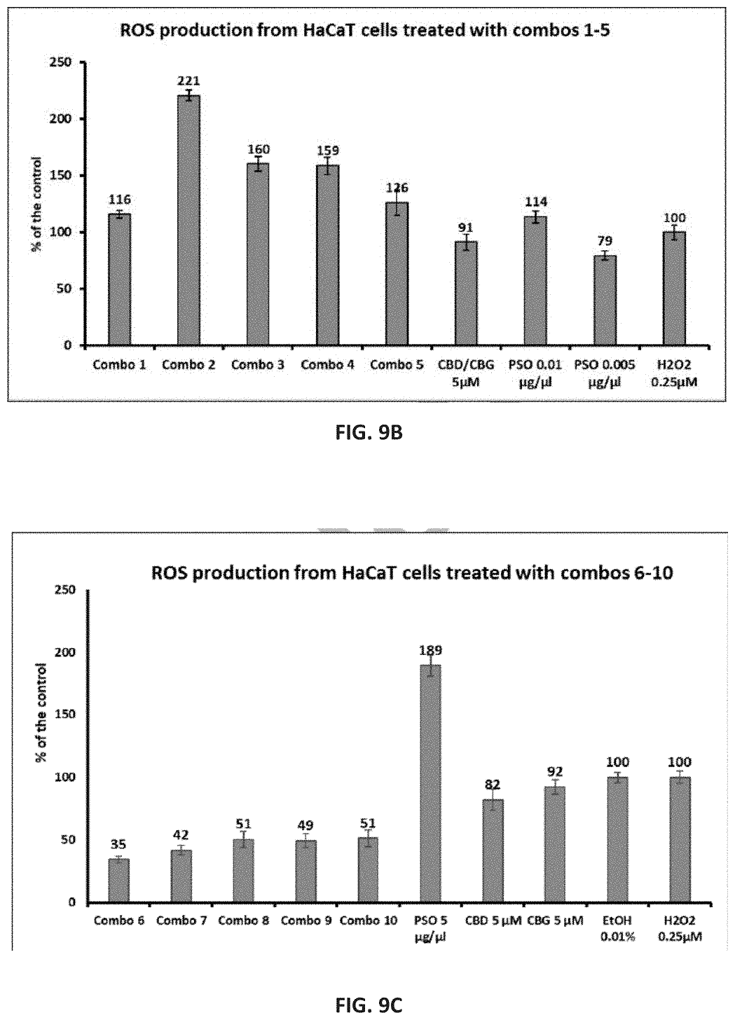

FIGS. 9A-C show the effect of various concentrations of CBD, CBG or PSO (FIG. 9A), of sample combinations 1-5 (FIG. 9B) and of sample combinations 6-10 (FIG. 9C) on ROS production from HaCaT cells.

Evaluating the effect of each of the tested samples relative to the solvent (ethanol) shows a pro-oxidative effect exhibited by PSO, and by sample combinations 1-5, and an anti-oxidative effect exhibited by all of sample combinations 6-10.

The pro-oxidative effect exhibited by sample combinations 1-5 and the very mild effect exhibited by CBD and CBG, put forward a promising effect of sample combinations 6-10.

### Example 3

### Intermediate Remarks

The assays described in Example 2 hereinabove tested the effect of each component alone (also referred to herein as “isolate” or “isolated sample”) and of various combinations of the tested components, on cells viability, inflammation and oxidation, in order to arrive at combination samples that exhibit non-toxicity and preferably pro-proliferative activity, along with anti-inflammatory and anti-oxidative effects.

The data obtained in these data indicate the following intermediate understandings with regard to the tested compositions:

Toxicity to Keratinocyte Cells:

A high concentration (e.g., 40 μM of CBD) is toxic to keratinocyte cells (see, FIG. 8A), and therefore lower concentrations of CBD should be used; CBG exhibits mild toxicity for keratinocyte cells only at a high concentration (see, FIG. 8A);

PSO at a concentration of 10 μg/μl and higher exhibits mild toxicity for keratinocyte cells (see, FIG. 8A).

Anti-Oxidative Activity:

PSO, at 5, 10 or 20 μg/μl, is pro-oxidative when used alone (see, FIGS. 9A-C), but when combined at low concentrations (lower than 5 μg/μl) with CBD and CBG, the combination samples exhibit the highest anti-oxidation activities, compared to combination samples with higher concentrations of PSO (see, FIG. 9C compared to FIG. 9B);

These data puts forward a beneficial effect of including PSO at a concentration lower than 5 μg/μl in combination with CBD and CBG at various concentrations.

Reference can also be made in this regard when comparing combos 4 and 5 (FIG. 9B) with combos 9 and 10 (FIG. 9C), respectively. The only difference in these combination samples is the amount of PSO, yet it can be seen that when a lower concentration of PSO is used, the pro-oxidative effect observed for combos 4 and 5 turns into an anti-oxidative effect for combos 9 and 10.

Taken together with the toxic effect of high concentration of CBD and mild toxicity of high concentration of CBG, these data indicate that a combination of CBD and CBG at non-toxic concentrations, at which anti-oxidation activity is mild when used alone, results in substantially enhanced anti-oxidation activity and emphasizes the beneficial effect of the combination of the present embodiments.

Anti-Inflammatory Activity:

An effect of the lower concentration of PSO can also be seen when comparing the data obtained for IL-6 secretion with combos 4 and 5 (FIG. 6A) with 9 and 10 (FIG. 7A), with the latter exhibiting higher anti-inflammatory effect.

From combos 6-10, which exhibited the highest anti-oxidation activity, combos 7 and 10 exhibited also the highest reduction in IL-6 secretion, indicating an anti-inflammatory activity.

### Example 4

### Exemplary Formulations and Products

A gel formulation comprising from 0.5 to 1% (e.g., 0.8-0.9%) by weight of a composition according to the present embodiments is prepared by mixing the composition with a gel carrier, optionally comprising additional agents as described herein in any of the respective embodiments.

An intense cream formulation comprising 1-10% (e.g., 2-5%; or about 4%) by weight of a composition according to the present embodiments is prepared by mixing the composition with a cream carrier, optionally comprising additional agents as described herein in any of the respective embodiments.

A lotion formulation comprising 0.1-0.5% (e.g., 0.2-0.3%) by weight of a composition according to the present embodiments is prepared by mixing the composition with a lotion carrier, optionally comprising additional agents as described herein in any of the respective embodiments.

A mask formulation comprising 1-5% (e.g., 3-4%) by weight of a composition according to the present embodiments is prepared by mixing the composition with a mask carrier, optionally comprising additional agents as described herein in any of the respective embodiments.

An oil formulation comprising 0.1-6% (e.g., 0.1-5, or 1-5, or 4-5, or 0.5-1%) by weight of a composition according to the present embodiments is prepared by mixing the composition with an oil carrier, optionally comprising additional agents as described herein in any of the respective embodiments. An exemplary representative deep cleansing oil formulation according to some embodiments of the present comprises 4-5% by weight of a composition according to the present embodiments. In exemplary embodiments, PSO is in an amount of about 4% by weight; CBD is in an amount of 0.1-0.5% by weight; and CBG is in an amount of 0.05-0.2% by weight. In exemplary embodiments, a weight ratio of CBD to CBG ranges from 1:1 to 5:1. In exemplary embodiments, a weight ratio of PSO to CBD ranges from 10:1 to 50:1. In exemplary embodiments, a weight ratio of CBD to CBG ranges from 1:1 to 5:1 and a weight ratio of PSO to CBD ranges from 10:1 to 50:1. In some of any of these exemplary embodiments, the formulation further comprises an oily carrier (containing pharmaceutically acceptable oil substances and glycerides) and additional components such as surface active agents, penetration enhancers, anti-oxidants, and the like.

A cream formulation comprising 0.1-2% (e.g., about 0.5, 1 or 1.5%) by weight of a composition according to the present embodiments is prepared by mixing the composition with a cream carrier, optionally comprising additional agents as described herein in any of the respective embodiments. Exemplary representative cream formulations such as face cream or a night cream according to some embodiments of the present comprise about 1.5% by weight of a composition according to the present embodiments. In exemplary embodiments, PSO is in an amount of about 0.5% by weight; CBD is in an amount of 0.9-1% by weight; and CBG is in an amount of 0.01-0.1% by weight. In exemplary embodiments, a weight ratio of CBD to CBG ranges from 5:1 to 10:1. In exemplary embodiments, a weight ratio of PSO to CBD ranges from 2:1 to 1:2. In exemplary embodiments, a weight ratio of CBD to CBG ranges from 5:1 to 10:1 and a weight ratio of PSO to CBD ranges from 2:1 to 1:2. In some of any of these exemplary embodiments, a cream formulation further comprises an aqueous phase (containing, for example, water and glycerin) in an amount of 50-70% by weight, an oily phase (containing pharmaceutically acceptable oil substances and plant extracts) in an amount of 20-30% by weight, and additional components such as surface active agents, emulsifiers, penetration enhancers, anti-oxidants, and the like. A serum formulation comprising 0.1-5% (e.g., 0.1-1 or 1-5%) by weight of a composition according to the present embodiments is prepared by mixing the composition with a serum carrier, optionally comprising additional agents as described herein in any of the respective embodiments. An exemplary representative serum formulation according to some embodiments of the present comprises 3-4% by weight of a composition according to the present embodiments. In exemplary embodiments, PSO is in an amount of about 2% by weight; CBD is in an amount of 1-2% by weight; and CBG is in an amount of 0.05-0.1% by weight. In exemplary embodiments, a weight ratio of CBD to CBG ranges from 5:1 to 30:1, or from 10:1 to 30:1, or from 10:1 to 20:1. In exemplary embodiments, a weight ratio of PSO to CBD ranges from 2:1 to 1:2. In exemplary embodiments, a weight ratio of CBD to CBG ranges from 5:1 to 30:1, or from 10:1 to 30:1, or from 10:1 to 20:1 and a weight ratio of PSO to CBD ranges from 2:1 to 1:2. In some of any of these exemplary embodiments, the formulation further an oily carrier (containing pharmaceutically acceptable oil substances and/or plant extracts) and additional components such as surface active agents, penetration enhancers, anti-oxidants, and the like.

The formulations were prepared using procedures known and acceptable in the art of cosmetic and cosmeceutical products.

The formulations are tested in acceptable dermatological assays to confirm the dermatological activity.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A cosmetic or cosmeceutical formulation comprising cannabidiol (CBD), an isolated cannabigerol (CBG) and pomegranate seed oil (PSO), wherein:
said CBD is an isolated CBD and said CBG is an isolated CBG; and/or
a concentration of said PSO in the formulation is no more than 1,000-folds, or no more than 500-folds, or no more than 100-folds, or no more than 50-folds, the concentration of said CBD or CBG.

2. The cosmetic or cosmeceutical composition of claim 1, wherein said CBD is an isolated CBD and said CBG is an isolated CBG.

3. The cosmetic or cosmeceutical formulation of claim 1, wherein an amount of PSO ranges from 0.01 to 5, or from 0.1 to 4, or from 0.1 to 2, % by weight of the total weight of the formulation.

4. The cosmetic or cosmeceutical formulation of claim 1, wherein an amount of CBD ranges from 0.1 to 2, % by weight of the total weight of the composition.

5. The cosmetic or cosmeceutical formulation of claim 1, wherein an amount of CBG ranges from 0.01 to 2, or from 0.01 to 1, % by weight of the total weight of the composition.

6. The cosmetic or cosmeceutical formulation of claim 1, wherein a weight ratio of CBD to PSO ranges from 2:1 to 1:100, or from 2:1 to 1:50, or from 1:1 to 100:1.

7. The cosmetic or cosmeceutical formulation of claim 1, wherein a weight ratio of CBG to PSO ranges from 1:5 to 1:100, or from 1:5 to 1:50.

8. The cosmetic or cosmeceutical formulation of claim 1, wherein a weight ratio of CBD to CBG ranges from 10:1 to 1:1, or from 10:1 to 2:1.

9. The cosmetic or cosmeceutical formulation of claim 1, wherein a weight ratio of CBD and CBG ranges from 2:1 to 10:1 and a weight ratio of PSO and CBD ranges from 1:5 to 50:1, or from 1:2 to 20:1.

10. The cosmetic or cosmeceutical formulation of claim 1, further comprising a cosmetically and/or cosmeceutically acceptable carrier.

11. The cosmetic or cosmeceutical formulation of claim 10, being in a form of a cream, an ointment, a paste, a gel, a lotion, a milk, an oil, a suspension, a solution, an aerosol, a spray, a foam, a powder (e.g., a pressed powder or a loose powder), a serum or a mousse.

12. The cosmetic or cosmeceutical formulation of claim 10, wherein a total concentration of said PSO, CBD and CBG ranges from 0.1 to 50, or from 1 to 50, % by weight of the total weight of the formulation.

13. A cosmetic or cosmeceutical product comprising the cosmetic or cosmeceutical formulation of claim 1.

14. The product of claim 13, being a skin care product, a make-up product, a men's grooming product, a sunscreen product, a female hygiene product, an oral cavity-care product or a hair care product.

15. The product of claim 13, being an anti-aging product, an anti-acne product, an anti-diaper rush product, a shampoo, a body lotion, a body cream, a face cream, a face lotion, a face mask, a body and/or face wash product, a cleansing product (for hair and/or skin and/or mucosal tissue such as oral cavity or vagina), a hygienic product, an anti-pigmentation product, a make-up product, an anesthetic product, a sunscreen product.

16. A method of treating, reducing or preventing (protecting from) a damage to a keratinous tissue or mucosal tissue, the method comprising topically applying to the keratinous or mucosal tissue the formulation of claim 1.

17. The method of claim 16, wherein the keratinous tissue is a facial tissue, a hair tissue, or a skin tissue.

18. The method of claim 16, wherein the mucosal tissue is a vaginal tissue.

19. The method of claim 16, wherein the mucosal tissue is in the oral cavity.

20. The method of claim 16, wherein said damage is associated with aging.

21. The method of claim 16, wherein said damage is associated with a dermatological condition.

22. The method of claim 21, wherein said damage is associated with skin inflammation.

23. The method of claim 21, wherein said dermatological condition is selected from atopic dermatitis, psoriasis, pigmentation, acne, eczema, xerosis, ichthyosis, keratosis, keratoderma, pruritus, dermatitis, neuro-dermatitis, dermatitis herpetiformis, actinic keratosis, hyper and inflamed keratosis, atopic eczema, melanoma, rosacea, urticaria, seborrheic dermatitis, skin cancer, and xeroderma pigmentosum.
